(19) United States
(12) Patent Application Publication
(10) Pub. No.: US 2020/0275931 A1
(43) Pub. Date: Sep. 3, 2020

(54) METHODS OF WOUND CLOSURE AND TREATMENT

(71) Applicant: ZIPLINE MEDICAL, INC., CAMPBELL, CA (US)

(72) Inventors: Amir Belson, Savyon (IL); Kei Ichiryu, Campbell, CA (US); Daren Stewart, Belmont, CA (US); Eric Storne, Menlo Park, CA (US)

(21) Appl. No.: 16/764,125

(22) PCT Filed: Nov. 16, 2018
(86) PCT No.: PCT/US2018/061482

§ 371 Date: May 14, 2020

Related U.S. Application Data
(60) Provisional application No. 62/588,169, filed on Nov. 17, 2017.
(60) Provisional application No. 62/722,467, filed on Aug. 24, 2018.

(51) Int. Cl. A61B 17/08; A61F 13/00

(52) U.S. Cl. CPC A61B 17/085; A61B 2017/00889; A61B 2017/081; A61F 13/00063

(57) ABSTRACT

Dry anti-microbial powder is applied to an incision or wound and surrounding area. A skin closure device is applied to the incision or wound. The incision or wound is closed, either before the closure device is applied or with closure device application. A sterile island dressing is then be applied over the incision or wound with the closure device and anti-microbial powder applied. The absorbent pad of the dressing can be applied directly over the incision or wound to absorb excess blood, pus, or other exudate. The anti-microbial powder mixes with the exudate before or as the exudate is absorbed. The anti-microbial powder in the exudate mixture reduces bacterial and/or microbial growth in the mixture, thereby reducing the infection risk to the closed incision or wound, as well as reducing fouling of the wound dressing and undesirable smells.

### CROSS-REFERENCE

This PCT application claims priority to U.S. Provisional Application No. 62/588,169, filed Nov. 17, 2017, and U.S. Provisional Application No. 62/722,467, filed Aug. 24, 2018, the contents of which are entirely incorporated herein by reference.

### BACKGROUND

The present disclosure relates generally to medical methods and apparatuses. More particularly, the present invention relates to methods and apparatuses for closing and treating surgical incisions or wounds.

Surgical closure devices based on adhesive based patches with right and left panels and lateral closure elements are known. Examples of such surgical closure devices include the Zip® line of surgical skin closure products available from Zipline Medical, Inc. of Campbell, Calif.. These skin closure products can facilitate the healing of wounds and incisions while minimizing scarring and eliminating the use of sutures and/or staples, which may cause additional tissue damage, lead to additional scarring, involve repeated visits to the clinic, or are in other ways less than ideal.

These skin closure products include an adhesive patch that can be placed over a patient's skin at a site where it is desired to form a surgical incision. After the patch is placed, an incision may be formed along an axial line extending through the middle of the patch. After the incision is formed, the incision can be opened to perform a desired procedure, and after the procedure is completed, the incision may be closed by drawing the inner edges of the panels together with the lateral ties of the closure device. Alternatively, the surgical skin closure products are placed over the patient's skin at a site where there is an existing wound or incision. In either case, an island dressing may be placed over the wound or incision closed with the closure device. The island dressing can, among other things, prevent further damage to the wound or incision, reduce infection risk to the wound or incision, assist in retaining moisture to provide a more ideal environment for healing, and/or absorb excess blood, pus, and/or other exudate from the wound or incision. Nevertheless, it may be further desired to provide improvements to meet the aforementioned purposes.

Alternatively or in combination to using an island dressing, a liquid adhesive may be placed over the wound or incision and allowed to set, thereby providing an additional structure to hold the closed wound or incision stationary, as well as for any of the aforementioned purposes of the island dressing. A common liquid adhesive for wound closure is cyanoacrylate (CA) and its various derivatives, and it is often used alone or with a mesh to secure a wound or incision. The use of a combination of the cyanoacrylate and mesh often forms a rigid assembly of the mesh and solidified adhesive that may be relatively inflexible and brittle, compared to, for example, the native skin and mesh without any adhesive. This rigidity and brittleness may lead to further damage of the wound or incision, such as when the wound or incision or skin nearby is exposed to lateral forces. This damage may include skin blistering and undesired exposure of the wound or incision to the external environment. Hence, it may be further desirable to provide improved closure elements and protocols to provide a more ideal environment for healing.

The following U.S. Patent Application Publications may be of interest: 2018/085260, 2017/0231822, 2017/0143341, 2017/0042541, 2017/0035422, and 2016/0310140.

The following U.S. Patents may be of interest: U.S. Pat. Nos. 10,004,485, 9,801,632, 9,789,005, 9,642,621, 9,561,034, 9,554,799, 9,089,328, 9,050,086, 9,764,055, 9,028,529, 8,663,275, 8,383,092, 8,252,335, 8,124,826, 8,118,792, 8,105,353, 7,267,681, 7,044,982, 6,899,104, 6,582,713, 6,566,577, 6,007,564, 5,976,117, 5,960,795, 5,947,998, 5,445,597, 5,116,620, 5,052,381, 4,976,726, 3,698,395, and 3,528,426.

### SUMMARY

The present disclosure relates to methods and apparatuses for closing and treating surgical incisions or wounds. A dry anti-microbial powder may be applied to an incision or wound, such as by sprinkling or daubing the powder on the incision or wound and surrounding area, or by spraying the powder over the incision or wound and surround area, for example, with a compressed gas powder delivery bottle, or by spraying with a solution of the powder such as in a fast drying liquid like alcohol. A skin closure device may be applied to the incision or wound, either before or after the anti-microbial powder has been applied. The incision or wound may be closed, either before the skin closure device is applied or with skin closure device application. A sterile island dressing may then be applied over the incision or wound, which has the skin closure device applied to maintain skin closure and also has the dry anti-microbial powder applied. An absorbent pad of the island dressing may be applied directly over the incision or wound to absorb excess blood, pus, or other exudate from the incision or wound. The dry anti-microbial powder may protect the wound from infection risk and, if the incision or wound secretes exudate or other liquid secretions, then the dry anti-microbial powder may mix with the exudate before or as the exudate is absorbed by the island dressing. The anti-microbial powder in the exudate mixture can reduce bacterial and/or microbial growth in the mixture, thereby reducing the infection risk to the closed incision or wound, as well as reducing fouling of the wound dressing and undesirable smells. The island dressing and/or the closure device may be regularly replaced, though not necessarily concurrently, and additional dry anti-microbial powder may be applied with each island dressing replacement.

The present disclosure also provides systems, devices, and methods for treating an incision or wound on a skin of a subject by applying a mesh assembly before applying a liquid adhesive that is allowed to set. The mesh assembly can have an anisotropic stretchability and once the liquid adhesive is applied at set, can form a mesh and adhesive complex also having an anisotropic stretchability. The mesh and adhesive complex can protect the incision or wound as it heals and its anisotropic stretchability can allow the complex to stretch preferentially in desired directions, such as along the length of the wound or incision, while resisting stretching in others.

In one aspect, disclosed herein is a method of closing and treating an incision or wound on a skin of a subject, the method comprising: adhering a skin closure device to skin surrounding an incision or wound; manipulating a closure component of the closure device to bring sides of the incision or wound closer together or to apply a lateral compressive force to the incision or wound; applying an anti-microbial powder to the incision or wound and surrounding skin; and applying an absorbent dressing over the incision or wound having the anti-microbial powder applied and the skin closure device adhered to the skin surrounding the incision or wound, wherein the anti-microbial powder mixes into exudate from the incision and wound, and the exudate having the anti-microbial powder mixed therein is absorbed by the absorbent dressing.

In some embodiments, the anti-microbial powder comprises a dry anti-microbial powder. In some embodiments, the anti-microbial powder comprises one or more of a silver anti-microbial powder, a silver-based anti-microbial powder, another metal anti-microbial powder, another metal-based anti-microbial dry powder, or non-metal based anti-microbial dry powder.

In some embodiments, adhering the skin closure device to the skin surrounding the incision or wound comprises adhering a first panel of the skin closure device to the skin on a first lateral side of the incision or wound, and adhering a second panel of the skin closure device to the skin on a second lateral side of the incision or wound opposite the first lateral side. In some embodiments, manipulating the closure component of the closure device to bring the sides of the incision or wound closer together or to apply the lateral compressive force to the incision or wound comprises tightening one or more lateral ties of the closure device.

In some embodiments, the absorbent dressing comprises a non-adhesive, absorbent pad, and wherein applying the absorbent dressing over the incision or wound comprises positioning the non-adhesive, absorbent pad over the skin or incision. In some embodiments, the absorbent dressing is non-medicated. In some embodiments, at least a portion of the absorbent dressing is medicated. In some embodiments, the method herein comprises replacing the absorbent dressing after about 1 to 14 days.

In some embodiments, the anti-microbial powder is applied to the incision or wound and surrounding skin before the skin closure device is adhered to the skin surrounding the incision or wound. In some embodiments, the anti-microbial powder is applied to the incision or wound and surrounding skin after the skin closure device is adhered to the skin surrounding the incision or wound. In some embodiments, the anti-microbial powder is applied to the incision or wound and surrounding skin before and after the skin closure device is adhered to the skin surrounding the incision or wound.

In some embodiments, a liquid adhesive is applied to the incision or wound. In some embodiments, the liquid adhesive is applied before the skin closure device is adhered to the skin surrounding the incision or wound. In some embodiments, the liquid adhesive is applied after the skin closure device is adhered to the skin surrounding the incision or wound. In some embodiments, the liquid adhesive is applied before the anti-microbial powder is applied to the incision or wound and the surrounding skin, for example, before the liquid adhesive has set. In some embodiments, the liquid adhesive is applied after the anti-microbial powder is applied to the incision or wound and the surrounding skin.

In another aspect, disclosed herein is a method of treating an incision or wound on a skin of a subject, the method comprising: positioning a mesh assembly over a wound or incision, the mesh assembly having an anisotropic stretchability; applying a liquid adhesive over the adhered mesh assembly; and allowing the liquid adhesive to set to form a mesh and adhesive complex also having an anisotropic stretchability.

In some embodiments, the method herein comprises adhering the mesh assembly to skin adjacent the wound or incision prior to allowing the liquid adhesive to set. In some embodiments, adhering the mesh assembly to the skin comprises pressing an adhesive bottom surface of the mesh assembly to the skin. In some embodiments, the adhesive bottom surface comprises a hydrocolloid adhesive. In some embodiments, pressing the adhesive bottom surface of the mesh assembly to the skin comprises pressing a first side of the adhesive bottom surface to the skin on a first side of the wound or incision and pressing a second side of the adhesive bottom surface to the skin on a second side of the wound or incision.

In some embodiments, the method further comprises stretching the mesh assembly across the wound or incision after pressing the first side of the adhesive bottom surface to the skin and before pressing the second side of the adhesive bottom surface to the skin, thereby applying tension to the wound or incision after both the first and second sides are adhered to the skin. In some embodiments, the mesh assembly having the anisotropic stretchability has a first stretchability in a first direction and a different second stretchability in a second direction transverse to the first direction.

In some embodiments, the first direction is a longitudinal direction with respect to the mesh assembly and the second direction is a transverse direction with respect to the mesh assembly, and wherein the first stretchability is greater than the second stretchability. In some embodiments, the mesh assembly comprises a plurality of lateral cut-outs to provide the anisotropic stretchability. In some embodiments, the plurality of lateral cut-outs is positioned on only a single lateral side of the mesh assembly. In some embodiments, the plurality of lateral cut-outs is positioned on both lateral sides of the mesh assembly. In some embodiments, the lateral cut-outs on a first lateral side of the mesh assembly are staggered with respect to the lateral cut-out on a second lateral side of the mesh assembly. In some embodiments, the mesh assembly is a single continuous structure. In some embodiments, the mesh assembly is a discontinuous structure. In some embodiments, the mesh assembly comprises a series of mesh segments arranged in a longitudinal line.

In some embodiments, the mesh assembly is made of nylon, polyurethane, or other polymer. In some embodiments, the liquid adhesive comprises cyanoacrylate.

In some embodiments, positioning the mesh assembly over the wound or incision comprises positioning the mesh assembly as coupled with an applicator frame over the wound or incision before removing the support frame. In some embodiments, the method further comprises removing one or more liners from a bottom surface of the mesh assembly. In some embodiments, the anisotropic stretchability is provided by a weave pattern of the mesh assembly.

In some embodiments, the method further comprises applying a therapeutic agent to the wound or incision or skin adjacent thereto. In some embodiments, the therapeutic agent comprises an anti-microbial agent, an anti-bacterial agent, a hemostatic agent, or a pain-relieving agent. In some embodiments, the therapeutic agent comprises a dry anti-microbial powder. In some embodiments, the therapeutic agent is incorporated into the mesh assembly. In some embodiments, the therapeutic agent is incorporated into the liquid adhesive. In some embodiments, the therapeutic agent is applied to the wound or incision before the liquid adhesive is applied. In some embodiments, the therapeutic agent is applied to the wound or incision after the liquid adhesive is applied. In some embodiments, the therapeutic agent is applied to the wound or incision before the liquid adhesive has set.

In some embodiments, the method further comprises unpacking the mesh assembly from a sealed package. In some embodiments, the mesh assembly comprises a liquid or gas activated adhesive and unpacking the mesh assembly from the sealed package activates the liquid or gas activated adhesive.

In some embodiments, the method further comprises applying a dressing over the mesh and adhesive complex.

In yet another aspect, disclosed herein is an apparatus for treating an incision or wound on a skin of a subject, the apparatus comprising: a mesh assembly having an anisotropic stretchability and configured to be applied over the wound or incision prior to applying a liquid adhesive to from a mesh and adhesive complex also having an anisotropic stretchability. In some embodiments, the mesh assembly comprises an adhesive bottom surface. In some embodiments, the adhesive bottom surface comprises a hydrocolloid adhesive. In some embodiments, the mesh assembly having the anisotropic stretchability has a first stretchability in a first direction and a different second stretchability in a second direction transverse to the first direction. In some embodiments, the first direction is a longitudinal direction with respect to the mesh assembly and the second direction is a transverse direction with respect to the mesh assembly, and wherein the first stretchability is greater than the second stretchability.

In some embodiments, the mesh assembly comprises a plurality of lateral cut-outs to provide the anisotropic stretchability. In some embodiments, the plurality of lateral cut-outs is positioned on only a single lateral side of the mesh assembly. In some embodiments, the plurality of lateral cut-outs is positioned on both lateral sides of the mesh assembly. In some embodiments, the lateral cut-outs on a first lateral side of the mesh assembly are staggered with respect to the lateral cut-out on a second lateral side of the mesh assembly. In some embodiments, the mesh assembly is a single continuous structure. In some embodiments, the mesh assembly is a discontinuous structure. In some embodiments, the mesh assembly comprises a series of mesh segments arranged in a longitudinal line.

In some embodiments, the mesh assembly is made of nylon, polyurethane, or other polymer. In some embodiments, the liquid adhesive comprises cyanoacrylate.

In some embodiments, the apparatus herein further comprises an applicator frame removable coupled to the mesh assembly. In some embodiments, the apparatus further comprising one or more liners removably coupled to a bottom surface of the mesh assembly.

In some embodiments, the anisotropic stretchability is provided by a weave pattern of the mesh assembly. In some embodiments, a therapeutic agent is incorporated into the mesh assembly.

In some embodiments, the therapeutic agent comprises an anti-microbial agent, an anti-bacterial agent, a hemostatic agent, or a pain-relieving agent.

In yet another aspect, disclosed herein is a method of treating an incision or wound on a skin of a subject, the method comprising: positioning a mesh assembly over a wound or incision, the mesh assembly comprising a series of discontinuous mesh segments arranged in a longitudinal line; applying a liquid adhesive over the adhered mesh assembly; and allowing the liquid adhesive to set to form a mesh and adhesive complex, the complex having an anisotropic stretchability.

In some embodiments, the method further comprises adhering the mesh assembly to skin adjacent the wound or incision prior to allowing the liquid adhesive to set. In some embodiments, adhering the mesh assembly to the skin comprises pressing an adhesive bottom surface of the mesh assembly to the skin. In some embodiments, the adhesive bottom surface comprises a hydrocolloid adhesive. In some embodiments, pressing the adhesive bottom surface of the mesh assembly to the skin comprises pressing a first side of the adhesive bottom surface to the skin on a first side of the wound or incision and pressing a second side of the adhesive bottom surface to the skin on a second side of the wound or incision. In some embodiments, the method further comprises stretching the mesh assembly across the wound or incision after pressing the first side of the adhesive bottom surface to the skin and before pressing the second side of the adhesive bottom surface to the skin, thereby applying tension to the wound or incision after both the first and second sides are adhered to the skin.

In some embodiments, the mesh assembly is made of nylon, polyurethane, or other polymer. In some embodiments, the liquid adhesive comprises cyanoacrylate.

In some embodiments, positioning the mesh assembly over the wound or incision comprises positioning the mesh assembly as coupled with an applicator frame over the wound or incision before removing the support frame. In some embodiments, the method further comprises removing one or more liners from a bottom surface of the mesh assembly.

In some embodiments, the method further comprises applying a therapeutic agent to the wound or incision or skin adjacent thereto. In some embodiments, the therapeutic agent comprises an anti-microbial agent, an anti-bacterial agent, a hemostatic agent, or a pain-relieving agent. In some embodiments, the therapeutic agent comprises a dry anti-microbial powder. In some embodiments, the therapeutic agent is incorporated into the mesh assembly. In some embodiments, the therapeutic agent is incorporated into the liquid adhesive. In some embodiments, the therapeutic agent is applied to the wound or incision before the liquid adhesive is applied. In some embodiments, the therapeutic agent is applied to the wound or incision after the liquid adhesive is applied. In some embodiments, the therapeutic agent is applied to the wound or incision before the liquid adhesive has set.

In some embodiments, the method further comprises unpacking the mesh assembly from a sealed package. In some embodiments, the mesh assembly comprises a liquid or gas activated adhesive and unpacking the mesh assembly from the sealed package activates the liquid or gas activated adhesive.

In some embodiments, the method further comprises applying a dressing over the mesh and adhesive complex.

In yet another aspect, disclosed herein is an apparatus for treating an incision or wound on a skin of a subject, the apparatus comprising: a mesh assembly comprising a series of discontinuous mesh segments arranged in a longitudinal line. In some embodiments, the mesh assembly is configured to be applied over the wound or incision prior to applying a liquid adhesive to from a mesh and adhesive complex. In some embodiments, the complex has an anisotropic stretchability.

In some embodiments, the mesh assembly comprises an adhesive bottom surface. In some embodiments, the adhesive bottom surface comprises a hydrocolloid adhesive. In some embodiments, the mesh assembly is made of nylon, polyurethane, or other polymer. In some embodiments, the liquid adhesive comprises cyanoacrylate.

In some embodiments, the apparatus further comprises an applicator frame removable coupled to the mesh assembly.

In some embodiments, the apparatus further comprises one or more liners removably coupled to a bottom surface of the mesh assembly.

In some embodiments, a therapeutic agent is incorporated into the mesh assembly. In some embodiments, the therapeutic agent comprises an anti-microbial agent, an anti-bacterial agent, a hemostatic agent, or a pain-relieving agent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1E2 show a method of closing and treating an incision or wound with a combination of a closure device, anti-microbial agent, and an island dressing;

FIG. 1A1 shows a perspective view of an incision or wound on a skin of a subject;

FIG. 1A2 shows a section view of the incision or wound on the skin as shown by FIG. 1A1;

FIG. 1B1 shows a perspective view of the incision or wound with an anti-microbial dry powder applied;

FIG. 1B2 shows a section view of the incision or wound with an anti-microbial dry powder applied as in FIG. 1B1;

FIG. 1C1 shows a perspective view of the incision or wound with both a closure device and the anti-microbial dry powder applied;

FIG. 1C2 shows a section view of the incision or wound with both the closure device and the anti-microbial dry powder applied as in FIG. 1C1;

FIG. 1D1 shows a perspective view of the incision or wound, now closed, with both a closure device and the anti-microbial dry powder applied;

FIG. 1D2 shows a section view of the incision or wound, now closed, with both the closure device and the anti-microbial dry powder applied as in FIG. 1C1;

FIG. 1E1 shows a perspective view of an island dressing applied over the closed incision or wound as well as the closure device and anti-microbial dry powder applied thereon;

FIG. 1E2 shows a section view of an island dressing applied over the closed incision or wound as well as the closure device and anti-microbial dry powder applied thereon as in FIG. 1E1;

FIG. 2A shows a perspective view of a closure device comprising a continuous mesh assembly with staggered, lateral cutouts, according to embodiments of the present disclosure;

FIG. 2B shows a perspective view of a closure device comprising a discontinuous mesh assembly comprising a series of mesh segments, according to embodiments of the present disclosure;

FIG. 3 shows a perspective view of a closure device comprising a mesh assembly coupled to a removable applicator frame, according to embodiments of the present disclosure;

FIG. 4 shows a perspective view of a closure device comprising a mesh assembly as it is applied to skin, according to embodiments of the present disclosure;

FIG. 5 shows a perspective view of a closure device comprising a discontinuous mesh assembly as it is applied to skin with an applicator frame, according to embodiments of the present disclosure;

FIG. 6A shows a perspective view of a closure device comprising a continuous mesh assembly with staggered, lateral cutouts and two continuous lateral adhesive panels, according to embodiments of the present disclosure; and

FIG. 6B shows a perspective view of a closure device comprising a continuous mesh assembly with staggered, lateral cutouts and two discontinuous lateral adhesive panels, according to embodiments of the present disclosure.

### DETAILED DESCRIPTION

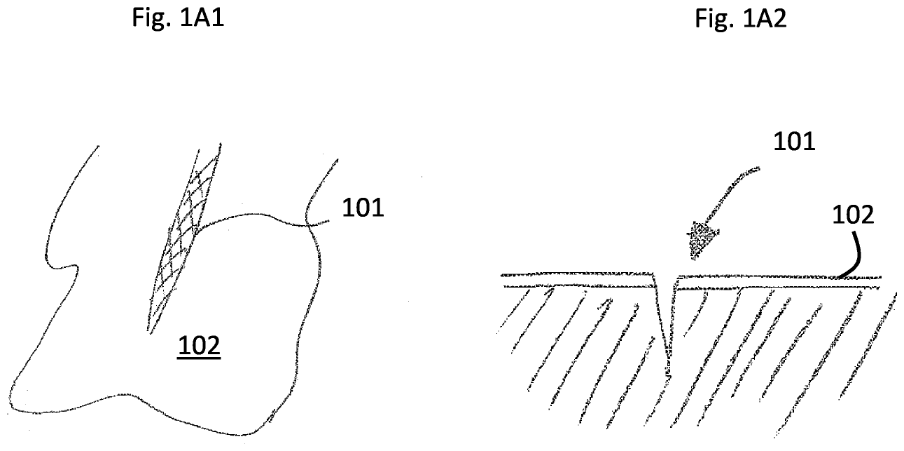

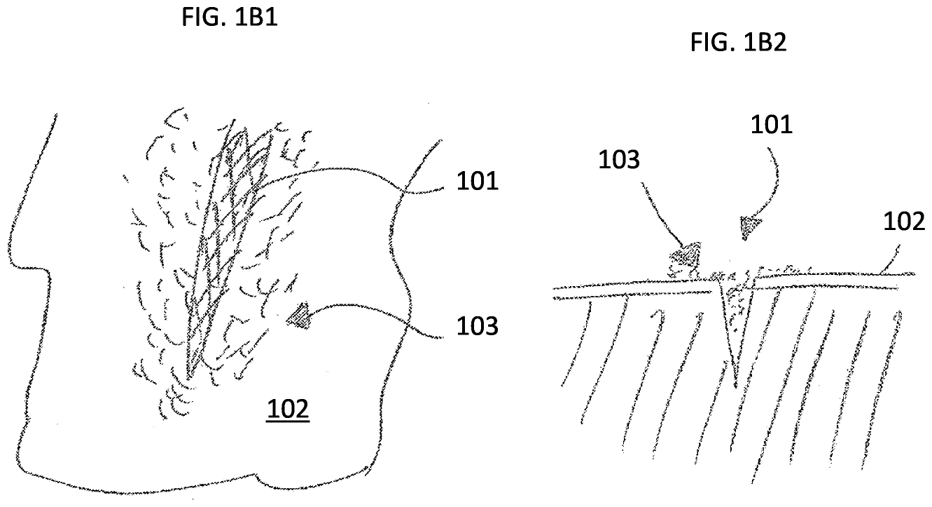

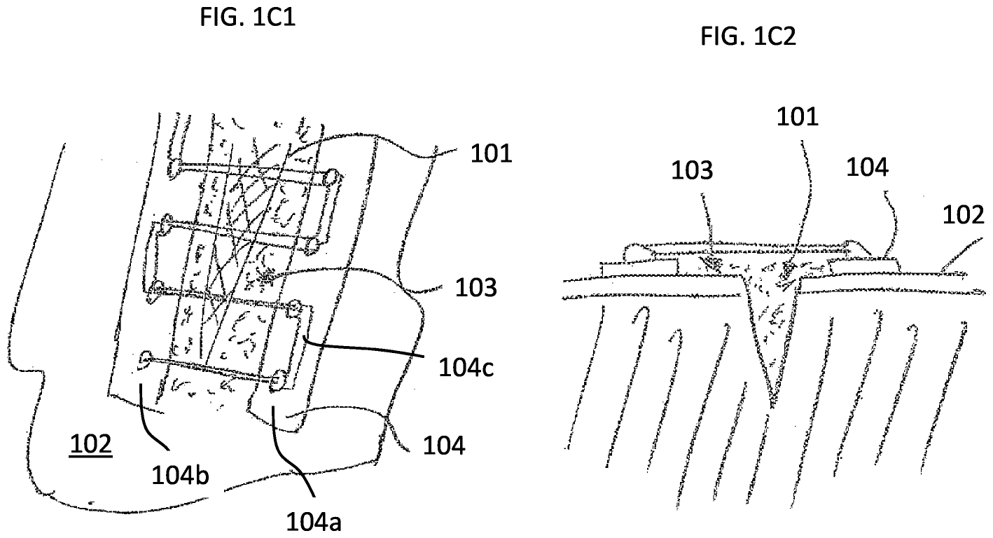

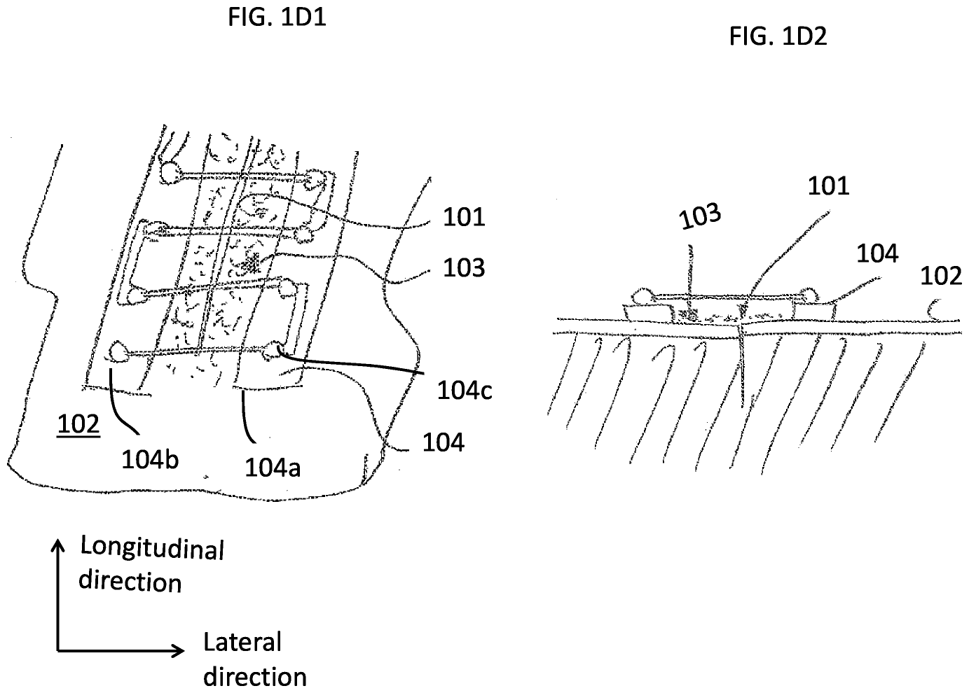

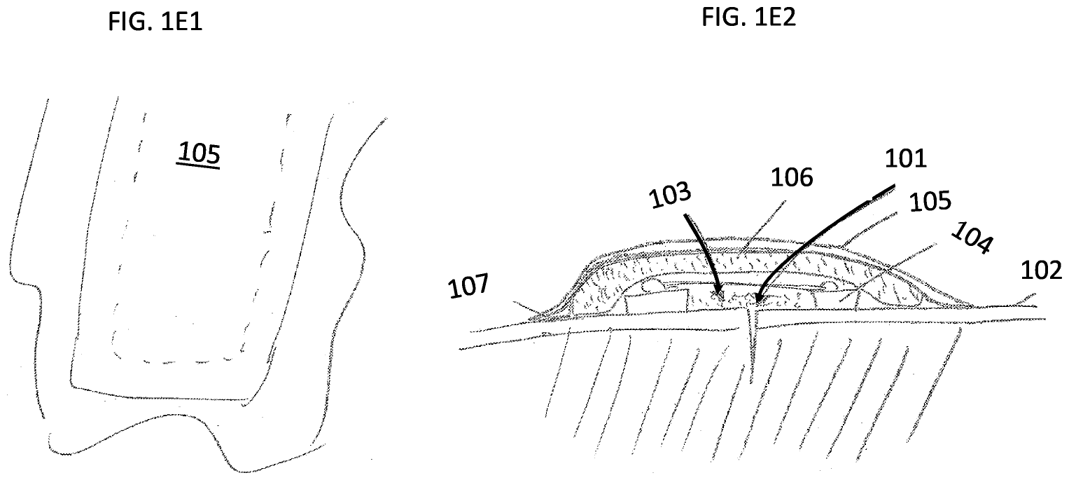

FIGS. 1A1-1E2 show exemplary embodiments of a method of closing and treating an incision or wound with a combination of a closure device, anti-microbial agent, and an island dressing.

FIGS. 1A1 and 1A2 show an incision or wound 101 on a skin 102 of a subject in a top view (FIG. 1A1) and cross-section view (FIG. 1A2). The incision or wound may be an incision resulting from a surgical procedure (e.g., a Caesarian section, a knee or other joint replacement surgery, a skin removal surgery, a biopsy, etc.) or from a non-surgical related injury.

FIGS. 1B1 and 1B2 show an anti-microbial dry powder 103 applied to the incision or wound 101 and surrounding area of FIGS. 1A1-1A2. The anti-microbial dry powder 103 may work directly on the incision or wound to reduce infection risk. In some embodiments, the anti-microbial dry powder 103 comprises a silver anti-microbial powder, a silver-based anti-microbial powder, another metal anti-microbial powder, another metal-based anti-microbial dry powder, non-metal based anti-microbial dry powder, or a combination thereof. Metal or metal-based anti-microbial powders may include those based on aluminum, antimony, arsenic, barium, bismuth, boron, copper, gold, lead, mercury, nickel, silver, thallium, tin, zinc, titanium, and any of their oxides, salts, esters, and other associated compounds. Non-metal based anti-microbial powders may include those based on various antimicrobial salts, oxides, saccharides, polysaccharides, silicates, etc. Further anti-microbial agents that may be used may include dry antibiotic medication in a powder form, dry polymers with anti-microbial activity, including: polymeric biocides, biocidal polymers, and biocide-releasing polymers, copper or sulfate based materials, sugars with anti-microbial activity and any other dry powder made from material with anti-microbial activity.

FIGS. 1C1 and 1C2 show a skin closure device 104 applied over the incision or wound 101 in a top view (FIG. 1C1) and cross-section view (FIG. 1C2). The skin closure device may be applied before and/or after the anti-microbial dry powder 103 is applied. The skin closure device may comprise two adhesive panels 104a, 104b, each applied to opposite lateral sides of the incision or wound 101, and a series of lateral ties or straps 104c. The lateral ties or straps 104c may be used to close the closure device 104 and/or the incision or wound 101, i.e., to pull the two panels laterally closer together to approximate the edges of the incision or wound. In many embodiments, the anti-microbial powder 103 migrates to the areas under the lateral ties or straps. Exemplary closure devices are described in granted U.S. Pat. Nos. 8,313,508, 8,323,313, 8,439,945, 9,050,086, 9,089,328, 9,179,914, 9,474,529, 9,554,799, 9,554,800, 9,561,034, 9,642,621, and 9,642,622, and pending U.S. patent application Ser. Nos. 13/665,160, 14/851,059 14/958,803, 14/958,818, 15/130,764, 15/201,088, 15/369,293, and 15/442,382. While the use of a surgical skin closure device along the likes of those available from Zipline Medical, Inc. of Campbell, Calif. is described, the methods described are applicable to any surgical skin closure device. These may include, for example, Steri-Strip® Elastic Skin Closures from The 3M Company of St. Paul, Minn., the DermaClip® product from DermaClip® US, LLC of Houston, Tex., various devices from the Dynamic Tissue Systems® products lines available from Southmedic, Inc. of Ontario, Canada, CURAD® Sterile Medi-Strips from Medline Industries, Inc. of Northfield, Ill., and butterfly closure strips available from various medical product suppliers, to name a few.

FIGS. 1D1 and 1D2 show the skin closure device 104 applied over the incision or wound 101 in a top view (FIG. 1D1) and cross-section view (FIG. 1D2), with the lateral ties 104c tightened to bring its two panels 104a, 104b laterally closer together and also closing the incision or wound, i.e., approximating its edges. Alternatively or in combination, the edges of the incision or wound may be approximated before the application of the skin closure device. In some embodiments, the lateral ties 104c of the skin closure device 104 may be tightened after being applied to a closed wound to further close the wound and add an additional lateral, compressive force, which may facilitate healing.

FIGS. 1E1-1E2 show an island dressing 105 applied over the closed incision or wound 101, including the closure device 104 and anti-microbial dry powder 103 applied thereon in a top view (FIG. 1E1) and cross-section view (FIG. 1E2). The island dressing 105 may comprise a non-adhesive absorbent pad 106 that may be placed directly over the incision or wound. The periphery of the island dressing may comprise an adhesive outer edge 107 to adhere to the skin and hold the island dressing in place. The island dressing may comprise a standard, commercially available island dressing available from the 3M Company of St. Paul, Minn., Cardinal Health, Inc. of Dublin, Ohio, and other medical product suppliers. As an alternative to island dressings, medical gauze in combination with medical tape may be used. While the use of a standard, un-medicated island dressing is described here, medicated island dressings may be used alternatively or in combination. The medication in these island dressings may be embedded in the dressing itself or may be applied to the dressing prior to its application to the incision or wound.

The absorbent pad 105 of the island dressing 105 absorbs excess blood, pus, or other exudate from the incision or wound 101. The dry anti-microbial powder 103 may mix with the exudate before or as the exudate is absorbed by the island dressing. The anti-microbial powder in the exudate mixture can reduce bacterial and/or microbial growth in the mixture, thereby reducing the infection risk to the closed incision or wound, as well as reducing fouling of the wound dressing 105 and undesirable smells. The island dressing 105 and/or the closure device 104 may be regularly replaced, though not necessarily concurrently, and additional dry anti-microbial powder 103 may be applied with each island dressing replacement. For example, the island dressing may be replaced after about 1 to 14 days depending on the amount of exudate that has been absorbed (i.e., if there has been a lot of exudate that has been absorbed, the island dressing may need to be replaced sooner.) In many embodiments, the closure device is not replaced at all except in cases where there are additional complications with the healing of the incision or wound. In many embodiments, additional dry anti-microbial powder may not need to be applied, such as if the amount of exudate from the incision or wound has become insignificant.

The present disclosure also provides systems, devices, and methods for treating an incision or wound on a skin of a subject by applying a mesh assembly before applying a liquid adhesive that is allowed to set. The mesh assembly may have an anisotropic stretchability and once the liquid adhesive is applied at set, may form a mesh and adhesive complex also having an anisotropic stretchability. The mesh and adhesive complex can protect the incision or wound as it heals and its anisotropic stretchability can allow the complex to stretch preferentially in desired directions, such as along the length of the wound or incision, while resisting stretching in others. The anisotropic stretchability may be provided by having cutouts on the mesh assembly, having the mesh assembly being partially or fully discontinuous, by varying the thickness of various regions of the mesh assembly, by provided different materials of different stretchability for different regions of the mesh assembly, by providing perforations or otherwise preferentially stretchable regions for the mesh assembly, and/or by otherwise patterning the outer periphery and/or the main body of the mesh assembly. In a similar manner, the mesh assembly may be provided with a vertical and/or lateral elasticity gradient to reduce adhesion loss and skin blistering in response to movements of tissue adjacent the adhered mesh assembly. Any of the anti-microbial and other therapeutic agents disclosed herein may be applied to the wound or incision directly or may be applied from the mesh assembly and/or liquid glue the agent may be incorporated into.

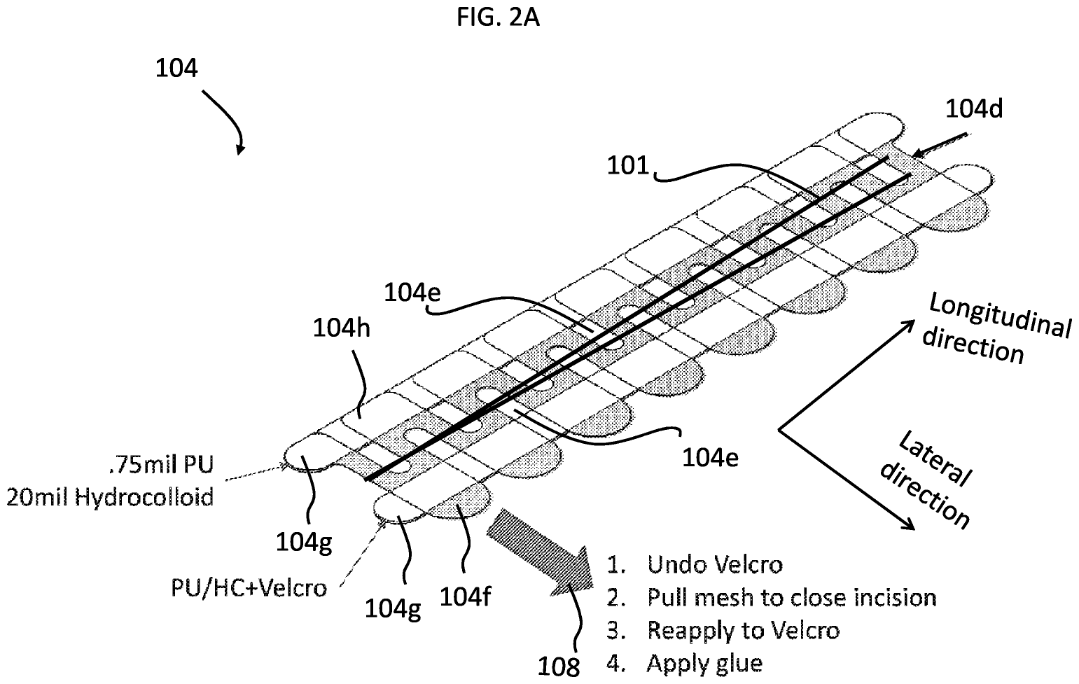

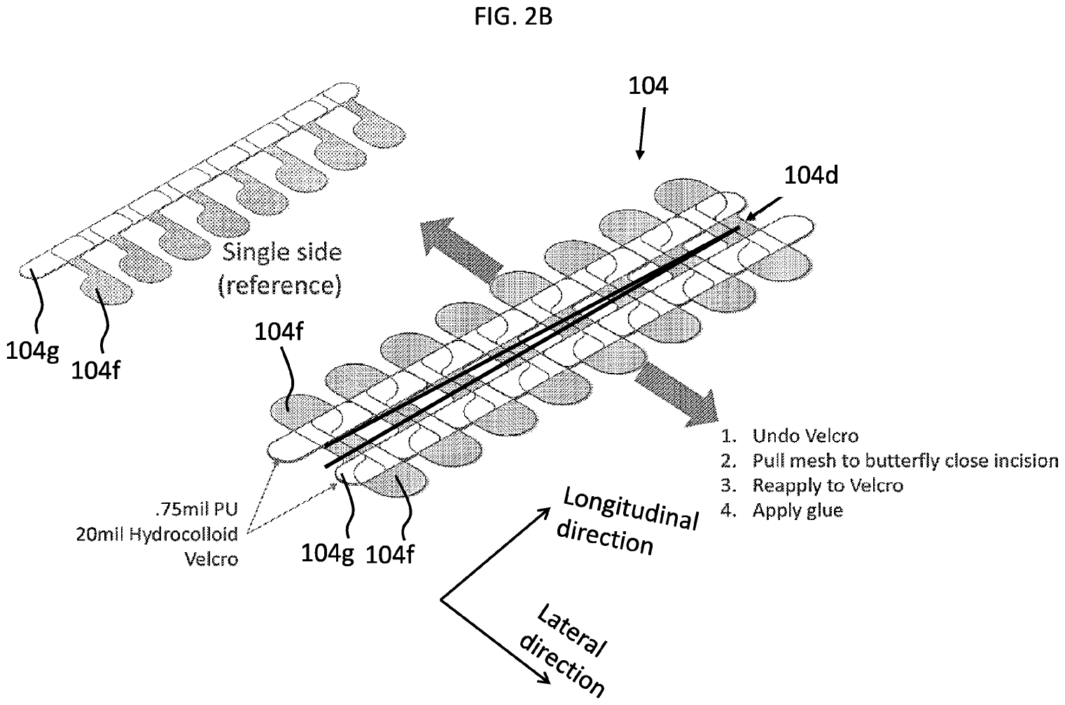

FIG. 2A shows an exemplary embodiment of a skin closure device 104 or equivalently herein, an apparatus for treating an incision or would comprising a continuous mesh assembly 104d with staggered, lateral cutouts 104e. The lateral cutouts traverse the width of the mesh (i.e., along the lateral direction) assembly only partially and are arranged in longitudinal series along the length of the mesh (i.e., along the longitudinal direction) assembly. The lateral cutouts 104e are on both lateral sides of the mesh assembly 104d and are staggered with respect to one another such that the mesh assembly has a sinusoidal configuration. In some embodiments, the later cutouts 104e can be placed such that the mesh assembly has other repetitive configuration along the longitudinal direction. One or both lateral sides of the mesh assembly may be enlarged at the ends of the curve of the sinusoid to form tabs 104f that a user can handle to apply the mesh assembly. In some embodiments, the tabs have different shapes or sizes from that shown in FIG. 2A.

The closure device 104 further comprises elongate panels 104g couple to either lateral side of the mesh assembly. The elongate panels 104g have a highly stretchable adhesive bottom layer, typically made of a hydrocolloid material. The adhesive bottom layer may be topped with a non-adhesive layer 104h, typically made of polyurethane or a similar polymer or material so that the upper surface of the closure device is not adhesive. One of the elongate panels 104g may be also topped with Velcro™ or a similar material to be removably coupled to the mesh assembly while the other elongate panel may be fixedly or irreversibly coupled to the mesh assembly. In some embodiments, both of the elongate panels may be topped with Velcro™ or similar material to be removably coupled to the mesh assembly as desired by the user.

To apply the closure device 104 to an incision or wound 101, liner(s) may be removed from the elongate panels 104 to expose their adhesive bottom layers. Then, the elongate panels 104g may be adhered to skin on either side of the wound or incision 101. One elongate panel may be adhered to the skin with the mesh assembly 104d coupled to the elongate panel. The other elongate panel may be (temporarily) separated from the mesh assembly. Once the elongate panel fixedly coupled to the mesh assembly is adhered to the skin, the mesh assembly 104d may be pulled across the wound or incision in the lateral direction 108, with their tabs 104f, for example, and then re-coupled to the other elongate panel topped with Velcro™ or a similar material. Since the attachment to the other elongate panel that can be (temporarily) separated from the mesh assembly is reversible, the attachment points of the mesh assembly to this elongate panel may be adjusted and re-adjusted as desired. Once the desired amount of lateral tension is applied to the incision or wound 101, liquid glue may be applied and allowed to set. In some embodiments, the liquid glue may be applied with an applicator having an accelerator for the drying of the liquid glue. The accelerator may be incorporated in the cone of the tube storing and dispensing the liquid glue.

FIG. 2B shows an embodiment of the skin closure device 104 comprising a discontinuous mesh assembly 104d comprising a series of mesh segments. In some embodiments, the series of mesh segments have substantially identical shapes and/or sizes. In some embodiments, the series of mesh segments are separated from each other so that the mesh assembly is discontinuous. In some embodiments, the mesh segments are connected via one or more elongate panel. Each mesh segment may have an enlarged end that may serve as tabs that a user can handle to apply the mesh assembly. Like the closure device of FIG. 2A, the closure device of FIG. 2B may further include elongate panels 104g that may be fixedly or removably attached to the mesh assembly 104d, may be made of similar materials, and may be applied in a similar manner.

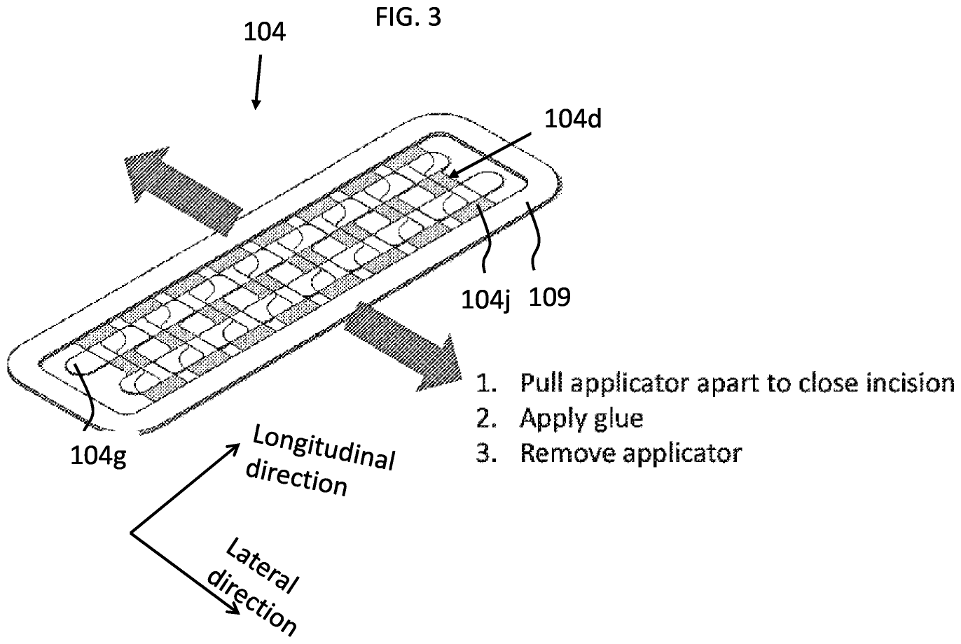

FIG. 3 shows a closure device 104 comprising a mesh assembly 104d coupled to a removable applicator frame 109. The mesh assembly of FIG. 3 may be similar to those of FIGS. 2A and 2B. Instead of using Velcro™ or similar material for selectively applying tension to portions of the mesh assembly, the lateral ends 104j of the mesh assembly may be removably coupled to the sides of the applicator frame 109. To apply the closure device 104, the applicator frame 109 may be pulled apart in the lateral direction to tension the mesh assembly before being adhered to the skin. The applicator frame 109 can be non-stretchable material or approximately non-stretchable material. The elongate panels 104g may be similar to those of FIGS. 2A and 2B and allow adhesion of the closure device 104 to the skin. After being adhered to skin, the mash assembly may have a tendency to reverse the tension toward the pre-tensioned stage thus enabling closure of the incision or wound. The liquid glue may then be applied to the open interior of the applicator frame and may be allowed to set before the applicator is removed from the mesh and adhesive complex. Alternatively, the liquid glue may be applied over the adhered mesh assembly after the applicator assembly is removed. In some embodiments, the applicator can be removed after the glue has been applied. In some embodiments, the applicator is attached to the mesh assembly via breakable connections so that the applicator can be removed from the mesh assembly or the rest of the closure device when a force applied on the applicator exceeds a threshold force. In some embodiments, the force needed to remove the applicator is much smaller than the adhesive force of the closure device to the skin so that removal of the applicator does not affect or change the closure device applied on the incision or cause discomfort to the subject.

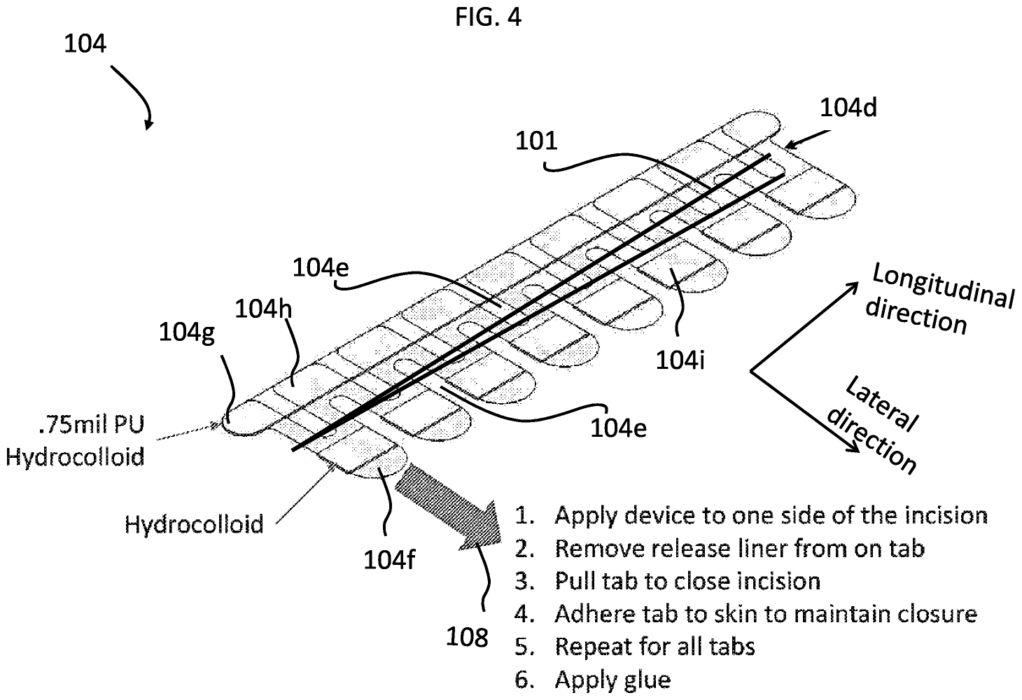

FIG. 4 shows a closure device 104 comprising a mesh assembly 104d with staggered, lateral cutouts much like the closure device of FIG. 2A. Similar to the closure device of FIG. 2A, the closure device of FIG. 4 may comprise an elongate panel 104g on one lateral side. On the other lateral side, segments 104i of an adhesive material such as hydrocolloid may be coupled to select portions of the mesh assembly 104d. Each of these adhesive segments may have their own individually removable liner. The closure device 104 of FIG. 4 may be applied similarly as the closure device of FIG. 2A. With the liner of the elongate panel 104g removed and the elongate panel then being adhered to one side of the wound or incision, one tab may be pulled to tension the respective portion of the mesh assembly and place it across the wound or incision 101 along the longitudinal direction, before its liner is removed and its respective adhesive segment 104i is adhered to the skin on the opposite side. This process may be repeated for the other tabs to fully adhere the mesh assembly to the skin before liquid adhesive is applied.

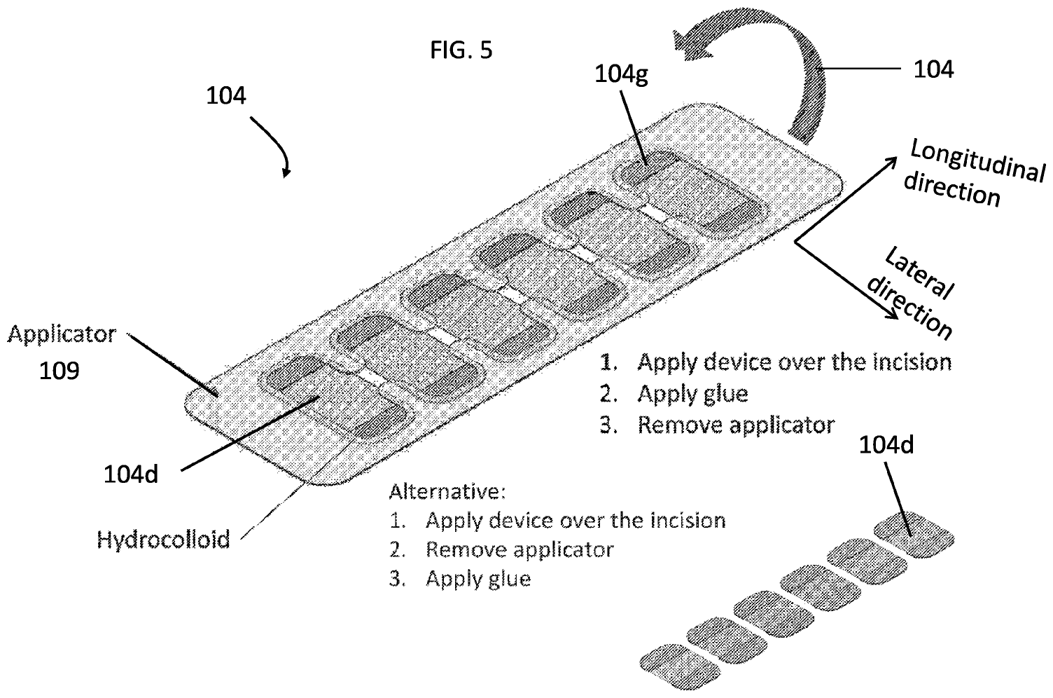

FIG. 5 shows a closure device 104 comprising a discontinuous mesh assembly 104d and an applicator frame 109 to apply tension to the lateral sides of each of the mesh segments of the mesh assembly. This closure device may be applied to a wound or incision similarly to the closure device of FIG. 3. For instance, the applicator 109 may be removed by being peeled away in the direction of arrow 110, leaving the adhesive panels 104g adhered to skin.

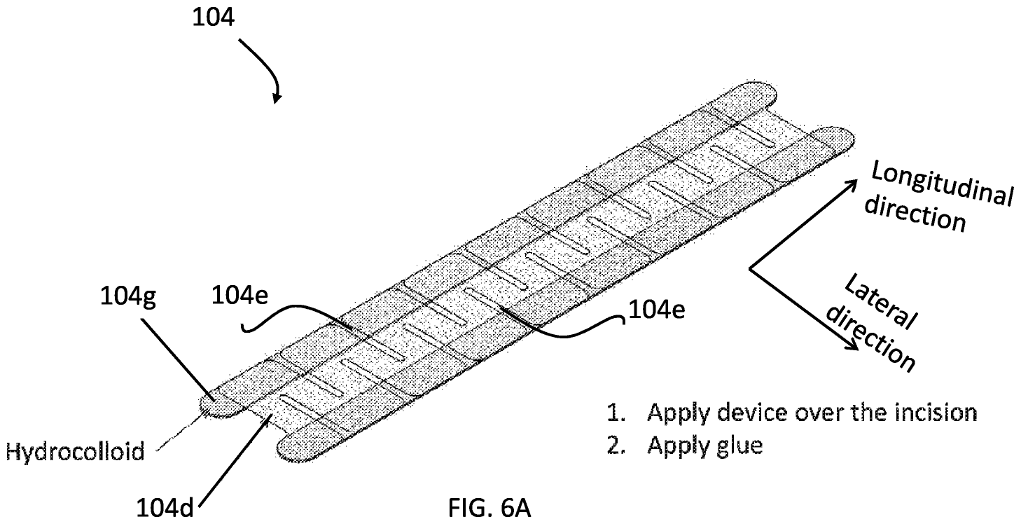

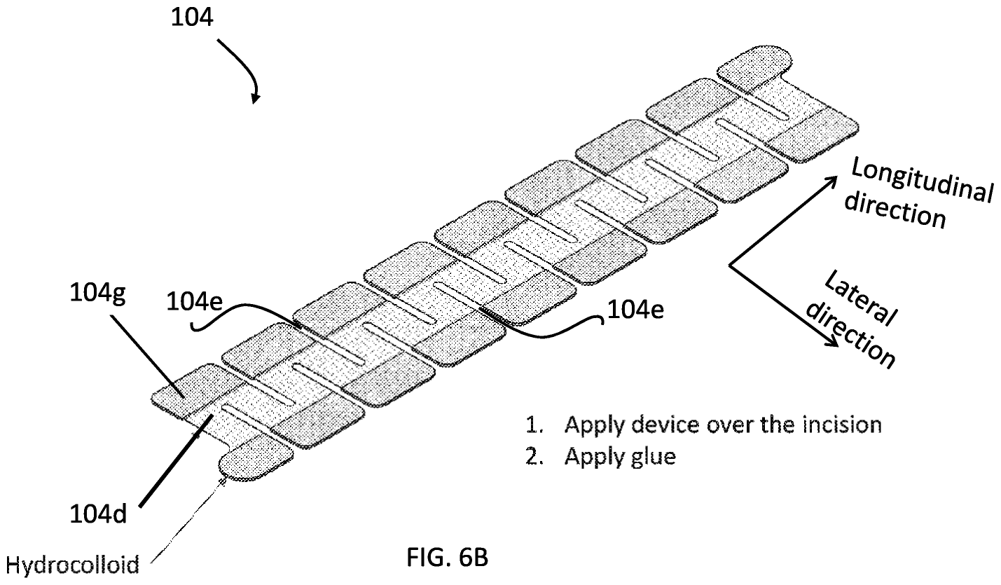

FIG. 6A shows a closure device 104 comprising a continuous mesh assembly 104d with staggered, lateral cutouts 104e similar to that of FIG. 2A. The closure device of FIG. 6A also comprises two continuous lateral adhesive panels 104g that may both be fixedly coupled to the mesh assembly. Instead of adhering both lateral panels to the skin first, one of the panels may be adhered to the skin first while the other still has its liner on. The mesh assembly can then be pulled and tensioned over the wound or incision before the remaining liner is removed and the remaining adhesive panel adhered to skin.

FIG. 6B shows a skin closure device 104 comprising a continuous mesh 104d assembly with staggered, lateral cutouts 104e and two discontinuous lateral adhesive panels 104g. The closure device of FIG. 6B may be similar to that of FIG. 6A, but may be applied by individually removing liner segments for each of the adhesive segments or panels 104g, rather than only two larger liners for each of the two continuous elongate panels as in the closure device of FIG. 6A.

As shown in FIGS. 2A-2B, FIGS. 3-5, and/or FIGS. 6A-6B, in some embodiments, the mesh assembly 104d includes a repetitive and evenly distributed pattern with cutouts along the longitudinal direction. In these embodiments, the adjacent cutouts are separated by a fixed distance along the longitudinal direction. Such evenly distributed pattern of cutouts can facilitate substantially uniform force/pressure application on different portion of the incision when the closure device is applied. In some embodiments, such evenly distributed pattern of cutouts can facilitate customization of the closure device to fit an incision or wound to be treated. In some embodiments, the mesh assembly includes a non-evenly distributed pattern with cutouts, for example, the distance between adjacent cutouts may be variable. Such variable pattern of cutout distribution in the closure device may facilitate optimization in force/pressure application for skin closure.

In some embodiments, the mesh assembly herein can be at least partially made of a flexible or stretchable material such as rubber, latex, polyurethane, silicone, polymer, or thermoplastic elastomer. In some embodiments, the mesh assembly includes degradable material that may be degraded or absorbed by the skin after a predetermined period time. In some embodiments, the mesh assembly is stretchable at least along the longitudinal direction. In some embodiments, the mesh assembly is stretchable at least along the lateral direction. In some embodiments, the mesh assembly, when stretched, can be at least 1.2, 1.5, 1.8, 2, 3, 4, 5, 6, 7, 8, 9, 10, or even more times of its unstretched size. In some embodiments, the stretchability of the mesh assembly is reversible when the force or pressure for stretching is removed so that it may facilitate optimal application of the closure device with the preferred stretched size and/or stretching direction(s). In some embodiments, the stretchable adhesive bottom layer of the elongate panel is stretchable at least along the lateral direction. In some embodiments, the bottom layer, when stretched, can be at least 1.2, 1.5, 1.8, 2, 3, 4, 5, 6, 7, 8, 9, 10, or even more times of its unstretched size. In some embodiments, the stretchability of the adhesive bottom layer is reversible.

In some embodiments, the skin closure device 104 or equivalently, the apparatus for treating an incision or wound on a skin of a subject can be of variable sizes depending on the size of the incision or wound need to be treated. For example, the skin closure device can be sized to enable skin closure and treat C-section incision. As another example, the skin closure device can be sized to enable skin closure with a cut on a figure tip. In some embodiments, the skin closure device can include a length along the longitudinal direction to be in the range of 3 mm to 0.5 meters.

In some embodiments, the skin closure device can include a length along the longitudinal direction to be in the range of 1 cm to 0.3 meters. In some embodiments, the skin closure device can include a length along the longitudinal direction to be in the range of 5 cm to 0.2 meters. In some embodiments, the skin closure device can include a length along the longitudinal direction to be in the range of 5 cm to 10 cm. In some embodiments, the skin closure device can include a length along the longitudinal direction to be in the range of 5 cm to 25 cm. In some embodiments, the skin closure device can include a length along the longitudinal direction to be in the range of 10 cm to 50 cm. In some embodiments, the width of the closure device along the lateral direction, before application, is in the range of 3 mm to 20 cm. In some embodiments, the width of the closure device along the lateral direction, before application, is in the range of 5 mm to 15 cm. In some embodiments, the width of the closure device along the lateral direction, before application, is in the range of 5 mm to 20 cm. In some embodiments, the width of the closure device along the lateral direction, before application, is in the range of 5 mm to 15 cm. In some embodiments, the width of the closure device along the lateral direction, before application, is in the range of 1 cm to 20 cm. In some embodiments, the width of the closure device along the lateral direction, before application, is in the range of 1 cm to 15 cm.

In some embodiments, the width of the cutouts along the lateral direction, before application, is in the range of 5 mm to 20 cm. In some embodiments, the width of the cutouts along the lateral direction, before application, is in the range of 5 mm to 15 cm. In some embodiments, the width of the cutouts along the lateral direction, before application, is in the range of 5 mm to 10 cm. In some embodiments, the width of the cutouts along the lateral direction, before application, is in the range of 8 mm to 15 cm. In some embodiments, the length of the cutouts along the longitudinal direction, before application, is in the range of 1 mm to 5 cm. In some embodiments, the length of the cutouts along the longitudinal direction, before application, is in the range of 1 mm to 4 cm. In some embodiments, the length of the cutouts along the longitudinal direction before application is in the range of 2 mm to 4 cm. In some embodiments, the length of the cutouts along the longitudinal direction, before application, is in the range of 3 mm to 3 cm.

In some embodiments, the width of the tabs, e.g., 104f, along the lateral direction, before application, is in the range of 1 mm to 5 cm. In some embodiments, the width of the cutouts along the lateral direction, before application, is in the range of 2 mm to 4 cm. In some embodiments, the width of the cutouts along the lateral direction, before application, is in the range of 2 mm to 3 cm. In some embodiments, the width of the cutouts along the lateral direction, before application, is in the range of 3 mm to 3 cm. In some embodiments, the length of the cutouts along the longitudinal direction, before application, is in the range of 5 mm to 10 cm. In some embodiments, the length of the cutouts along the longitudinal direction, before application, is in the range of 5 mm to 8 cm. In some embodiments, the length of the cutouts along the longitudinal direction before application is in the range of 5 mm to 6 cm. In some embodiments, the length of the cutouts along the longitudinal direction, before application, is in the range of 5 mm to 5 cm.

In some embodiments, the skin closure device can be pre-sized so that the user can select an appropriate size for a specific wound or incision. In some embodiments, the skin closure device can be customized by the user using a pre-sized closure device for optimal treatment of a wound or incision. For example, the user can cut the closure device shorter along the longitudinal direction to fit a wound to be treated. In some embodiments, the user can apply two or more closure devices in series with or without overlap to cover a wound or incision.

One or more of the components of the apparatuses or closure devices disclosed herein, including one or more of the mesh assemblies, elongate panels, the absorbent pad, etc. may be comprised of, be coated with, or otherwise incorporate one or more of an antifungal, antibacterial, antimicrobial, antiseptic, anti-inflammatory, or medicated material. For example, such materials may be incorporated into the hydrocolloid adhesive layer, as another layer or coating between the skin and the adhesive layer (covering at least a portion of the adhesive layer), incorporated into the base assembly cover or at least its adhesive layer, etc. One or more wells, grooves, openings, pores, or similar structures may be provided on the device or apparatus components to facilitate such incorporation. In many embodiments, such materials may comprise one or more of silver, iodide, zinc, chlorine, copper, or natural materials such as tea tree oil as the active agent. Examples of such antifungal, antibacterial, antimicrobial, antiseptic, anti-inflammatory, or medicated materials include, but are not limited to, the Acticoat™ family of materials available from Smith & Nephew plc of the U.K., the Acticoat® Moisture Control family of materials available from Smith & Nephew plc of the U.K., the Contreet® Foam family of materials available from Coloplast A/S of Denmark, the UrgoCell® Silver family of materials available from Urgo Limited of the U.K. (a subsidiary of Laboratoires URGO of France), the Contreet® Hydrocolloid family of materials available from Smith & Nephew plc of the U.K., the Aquacel® Ag family of materials available from ConvaTec Inc. of Skillman, N.J., the Silvercel® family of materials available from Kinetic Concepts, Inc. of San Antonio, Tex., Actisorb® Silver 220 available from Kinetic Concepts, Inc. of San Antonio, Tex., the Urgotul® SSD family of materials available from Urgo Limited of the U.K. (a subsidiary of Laboratoires URGO of France), the Inadine® family of materials available from Kinetic Concepts, Inc. of San Antonio, Tex., the Iodoflex® family of materials available from Smith & Nephew plc of the U.K., the Sorbsan Silver™ family of materials available from Aspen Medical Europe Ltd. of the U.K., the Polymem Silver® family of materials available from Ferris Mfg. Corp. of Burr Ridge, Ill., the Promogram™ family of materials available from Kinetic Concepts, Inc. of San Antonio, Tex., the Promogram Prisma™ family of materials available from Kinetic Concepts, Inc. of San Antonio, Tex., and the Arglaes® family of materials available from Medline Industries, Inc. of Mundelein, Ill. Examples of anti-inflammatory agents that may be used may include the class of topical non-steroidal anti-inflammatory agents (e.g., NSAIDs) such as diclofenac sodium, diclofenac potassium, and diclofenac diethylamine, to name a few. Components of the closure devices described in commonly owned U.S. Pat. Nos. 8,313,508, 8,323,313, and 8,439,945; U.S. Patent Publication No. 2013/0066365; and PCT application nos. US 2010/000430, US 2011/139912, US 2011/40213, US 2011/34649, and US 2013/067024 may also be comprised of, be coated with, or otherwise incorporate one or more of an antifungal, antibacterial, antimicrobial, antiseptic, anti-inflammatory, or medicated material, including but not limited to one or more of the materials listed above.

In many embodiments, topical medicinal agents are incorporated directly into the wound closure appliances described herein. Because a wound closure device is often applied in close proximity to a wound or incision in need of medicinal protection, the incorporation of such medicines directly into the closure device may be beneficial. In wounds at risk of infection, incorporation of anti-microbial agents may be beneficial, for example. Anti-microbial agents may include antibiotic medicines as well as antiseptic metal ions and associated compounds which may include silver, iodine, copper, and chlorine, or natural materials such as tea tree oil. In wounds prone to fungus, medicinal agents such as zinc may be warranted, for example. To facilitate healing, various anti-inflammatory agents (e.g., NSAIDs) may be incorporated into the closure device. Combinations of any of these agents may also be of benefit and thus may be incorporated into wound closure appliances.

Topical medicinal agents may be incorporated into the closure devices in a way to give the closure devices the ability to wick exudate away from the wound (e.g., to direct unwanted organisms away from the wound and/or prevent skin maceration), while keeping the wound sufficiently hydrated for improved healing.

In exemplary embodiments, a medicinal agent comprising a silver compound such as silver sulfate may be incorporated into one or more components of the closure devices or apparatuses herein.

The mesh assembly and the elongate panels of the incision closure devices described herein preferably may be covered with a flexible adhesive film material at the end of a wound or incision closure. This film area preferably may be larger than the elongate panel elements such that it overlaps the elements onto the skin. The film may help prevent migration of the closure device and may prevent any accidental movements of the closure device. The cover film may be made from stretchable materials like rubber, latex, polyurethane, silicone or thermoplastic elastomer, etc. In preferred embodiments, a thin cover (e.g., laminate of 0.001″ urethane and 0.002″ thick acrylic adhesive), can have a greater compliance than the composite structure of the base panel elements. As a result, the cover may offer some strain relief between the exposed skin and the mesh assembly. The cover may also be transparent to allow visual inspection of the incision. The cover may completely seal across the incision (e.g., as a barrier to infection) or there may be openings in the cover that are aligned with the incision line to allow passage of any exudates from the wound. The cover may also serve to improve the apposition of the incision edges by bridging the panels and adhering to the skin edges. The cover may also be constructed with additional reinforcing elements that improve the tensile strength between the panel elements but allow for compliance along the incision length. A preferred embodiment may comprise a series of polyethylene adhesive tape strips applied to the cover.

While the user may apply the cover after the mesh assembly and panels are placed on the skin, it is also conceived that the cover material may be supplied as a “skirt” extending around the outer perimeter of the mesh assembly. Thus, alignment of the cover materials relative to the closure device may not be dependent on the user placement. These same cover materials may provide the effects of preventing exposure of the hydrocolloid adhesive to patient clothing, limitation of migration of the hydrocolloid or other adhesive lower layer, providing strain relief for the tension on the mesh assembly, etc.

In many embodiments, a hydrocolloid adhesive is used for tensioning skin for incision closure. The hydrocolloid may be prevented from creep by one or more of (1) using a laminate on the surface to limit creep or (2) applying an adhesive cover across the skin and the hydrocolloid adhesive to prevent creep and to provide strain relief to the skin to prevent skin damage.

In many embodiments, a cover can be used with the closure devices herein, which may include one or more of perforations or openings to allow removal of wound exudate (as well as any applied bandages/absorbent material) without removal of the adhered mesh assembly.

In many embodiments, the cover comprises a composite of flexible urethane and reinforced strips. The composite construction may provide strength across the incision as well as provide for compliance along the incision length.

In many embodiments, the cover in combination with the mesh assembly aligns the skin incision edges or significantly prevents subsequent misalignment of the skin edges, in both the axial and lateral directions.

In many embodiments, cover liner configurations are provided such that part of the cover can be applied to the skin first, which then aids in the removal of other liners and thus may help control the thin materials so they lay out evenly with minimal wrinkling.

In many embodiments, the removal of a first liner may allow visualization during placement and may prevent the remainder of the device from sticking to the user.

Although the above steps describe and show particular methods of closing and treating an incision or wound in accordance with many embodiments, a person of ordinary skill in the art will recognize many variations based on the teaching described herein. The steps may be completed in a different order, steps may be added or omitted, some of the steps may comprise sub-steps, and many of the steps may be repeated as often as beneficial to the treatment.

Unless otherwise defined, all technical terms used herein have the same meaning as commonly understood by one of ordinary skill in the art to which this disclosure belongs. As used in this specification and the appended claims, the singular forms “a,” “an,” and “the” include plural references unless the context clearly dictates otherwise. Any reference to “or” herein is intended to encompass “and/or” unless otherwise stated. As used in this specification and the claims, unless otherwise stated, the term “about,” “substantially,” and “approximately” refers to variations of less than or equal to +/−1%, +/−2%, +/−3%, +/−4%, +/−5%, +/−6%, +/−7%, +/−8%, +/−9%, +/−10%, +/−11%, +/−12%, +/−14%, +/−15%, or +/−20% of the numerical value depending on the embodiment. As a non-limiting example, about 100 meters represents a range of 95 meters to 105 meters (which is +/−5% of 100 meters), 90 meters to 110 meters (which is +/−10% of 100 meters), or 85 meters to 115 meters (which is +/−15% of 100 meters) depending on the embodiments.

While preferred embodiments of the present disclosure have been shown and described herein, it will be obvious to those skilled in the art that such embodiments are provided by way of example only. Numerous variations, changes, and substitutions will now occur to those skilled in the art without departing from the scope of the present disclosure. It should be understood that various alternatives to the embodiments of the present disclosure described herein may be employed in practicing the inventions of the present disclosure. It is intended that the following claims define the scope of the invention and that methods and structures within the scope of these claims and their equivalents be covered thereby.

1. A method of closing and treating an incision or wound on a skin of a subject, the method comprising:
adhering a skin closure device to skin surrounding an incision or wound;
manipulating a closure component of the skin closure device to bring sides of the incision or wound closer together or to apply a lateral compressive force to the incision or wound;
applying an anti-microbial powder to the incision or wound and surrounding skin; and
applying an absorbent dressing over the incision or wound having the anti-microbial powder applied and the skin closure device adhered to the skin surrounding the incision or wound,
wherein the anti-microbial powder mixes into exudate from the incision and wound, and the exudate having the anti-microbial powder mixed therein is absorbed by the absorbent dressing.

2. (canceled)

3. The method of claim 1, wherein the anti-microbial powder comprises one or more of a dry anti-microbial powder, a silver anti-microbial powder, a silver-based anti-microbial powder, another metal anti-microbial powder, another metal-based anti-microbial dry powder, or non-metal based anti-microbial dry powder.

4. The method of claim 1, wherein adhering the skin closure device to the skin surrounding the incision or wound comprises adhering a first panel of the skin closure device to the skin on a first lateral side of the incision or wound, and adhering a second panel of the skin closure device to the skin on a second lateral side of the incision or wound opposite the first lateral side.

5. The method of claim 4, wherein manipulating the closure component of the skin closure device to bring the sides of the incision or wound closer together or to apply the lateral compressive force to the incision or wound comprises tightening one or more lateral ties of the skin closure device.

6-7. (canceled)

8. The method of claim 1, wherein at least a portion of the absorbent dressing is medicated.

9-12. (canceled)

13. The method of claim 1, further comprising applying a liquid adhesive to the incision or wound.

14-18. (canceled)

19. A method of treating an incision or wound on a skin of a subject, the method comprising:
positioning a mesh assembly over a wound or incision, the mesh assembly having an anisotropic stretchability;
applying a liquid adhesive over the adhered mesh assembly; and
allowing the liquid adhesive to set to form a mesh and adhesive complex, the adhesive complex also having an anisotropic stretchability.

20. The method of claim 19, further comprising adhering the mesh assembly to skin adjacent the wound or incision by pressing an adhesive bottom surface of the mesh assembly to the skin prior to allowing the liquid adhesive to set.

21-22. (canceled)

23. The method of claim 20, wherein pressing the adhesive bottom surface of the mesh assembly to the skin comprises pressing a first side of the adhesive bottom surface to the skin on a first side of the wound or incision and pressing a second side of the adhesive bottom surface to the skin on a second side of the wound or incision.

24. (canceled)

25. The method of claim 19, wherein the mesh assembly having the anisotropic stretchability has a first stretchability in a first direction and a different second stretchability in a second direction transverse to the first direction.

26. (canceled)

27. The method of claim 19, wherein the mesh assembly comprises a plurality of lateral cut-outs to provide the anisotropic strechability.

28-35 (canceled)

36. The method of claim 19, wherein positioning the mesh assembly over the wound or incision comprises positioning the mesh assembly as coupled with an applicator frame over the wound or incision before removing the a support frame.

37. The method of claim 19, further comprising removing one or more liners from a bottom surface of the mesh assembly.

38. (canceled)

39. The method of claim 19, further comprising applying a therapeutic agent to the wound or incision or skin adjacent thereto.

40-47. (canceled)

48. The method of claim 19, wherein the mesh assembly comprises a liquid or gas activated adhesive and unpacking the mesh assembly from a sealed package activates the liquid or gas activated adhesive.

49. The method of claim 19, further comprising applying a dressing over the mesh and adhesive complex.

50-68. (canceled)

69. A method of treating an incision or wound on a skin of a subject, the method comprising:
positioning a mesh assembly over a wound or incision, the mesh assembly comprising a series of discontinuous mesh segments arranged in a longitudinal line;
applying a liquid adhesive over the mesh assembly; and
allowing the liquid adhesive to set to form a mesh and adhesive complex, the adhesive complex having an anisotropic stretchability.

70. The method of claim 69, further comprising adhering the mesh assembly to skin adjacent the wound or incision prior to allowing the liquid adhesive to set.

71-76. (canceled)

77. The method of claim 69, wherein positioning the mesh assembly over the wound or incision comprises positioning the mesh assembly as coupled with an applicator frame over the wound or incision before removing a support frame.

79. The method of claim 69, further comprising applying a therapeutic agent to the wound or incision or skin adjacent thereto.

80-98. (canceled)
